(19) United States
(12) Patent Application Publication
(10) Pub. No.: US 2025/0369969 A1
(43) Pub. Date: Dec. 4, 2025

(54) Assessment of immune cells in the uterine fluid at the time of the embryo transfer

(71) Applicant: University of Miami, Miami, FL (US)

(72) Inventors: George Attia, Miami, FL (US); Natasa Strbo, Miami, FL (US)

(21) Appl. No.: 19/223,543

(22) Filed: May 30, 2025

Related U.S. Application Data
(60) Provisional application No. 63/653,492, filed on May 30, 2024.

(51) Int. Cl. G01N 33/569; A61B 10/04; G01N 33/543; G01N 33/68

(52) U.S. Cl. CPC G01N 33/56972; A61B 10/04; G01N 33/543; G01N 33/6872; G01N 2333/70503

(57) ABSTRACT

Disclosed is a method for frozen embryo transfer (FET) in a subject. The method comprises acquiring a uterine cavity fluid sample from the subject on the day of FET and performing one or more assays on the uterine cavity fluid sample to measure immune cell composition of the sample and determining if the sample comprises a composition of immune cells at a threshold value that indicates higher success of transfer implantation and transferring the embryo into the subject.

### CROSS-REFERENCE TO RELATED APPLICATION

This application claims the priority benefit of U.S. Provisional Patent Application No. 63/653,492, filed on May 30, 2024, the disclosure of which is hereby incorporated by reference in its entirety.

### FIELD OF DISCLOSURE

Inventions disclosed herein generally relate to the field of frozen embryo transfer. Specifically, disclosed herein are methods and systems for frozen embryo transfer and analysis of a sample taken from a subject on the day of transfer that indicate a likelihood of a higher or lower success of transfer implantation in the subject.

### BACKGROUND

Embryo implantation is one of the major steps of the human reproductive process [1]. Endometrial receptivity is a primary factor that influences the implantation of a naturally conceived pregnancy, as well as the implantation of an embryo transferred via assisted reproductive technology (ART). Implantation is a complex event that requires several processes to work in harmony in order for the embryo to implant into the endometrium [2]. Over the past several years, an effort has been made to elicit the various endometrial factors that are critical to the process of implantation. Many markers have been examined, including ultrasonographic, histological and molecular markers, such as interleukin-1 (IL-1), tumor necrosis factor-alpha (TNF-α), αvβ3 integrin, cell adhesion molecules (CAM), CD44, trophinin and cadherin-11 [3-6]. The endometrial receptivity analysis (ERA), a microarray-based machine learning predictive model that classifies the endometrium tissue based on its receptivity status, has been used to assist physicians in determining the optimal window of implantation (WOI). However, its validity has come into question in recent studies [6].

### SUMMARY

Disclosed is a method for frozen embryo transfer (FET) in a subject. The method comprises acquiring, on the day of FET, a uterine cavity fluid sample from the subject, performing one or more assays on the uterine cavity fluid sample to measure immune cell composition of the sample, determining if the sample comprises a percent composition of immune cells above or below a threshold value of immune cell composition to provide a determined threshold value that indicates higher success of transfer implantation, and transferring the frozen embryo into the subject if the sample comprises a percent composition of immune cells above a determined threshold value or a percent composition of immune cells below a threshold value that indicates higher success of transfer implantation.

In some embodiments, the uterine cavity fluid sample contains cells.

Also disclosed is a method for frozen embryo transfer (FET) in a subject, comprising acquiring, on the day of FET, a uterine cavity fluid sample from the subject, performing one or more assays on the uterine cavity fluid sample to measure immune cell composition of the sample, determining if the sample comprises a percent composition of gamma delta T cells above a threshold value and/or a percent composition of CD66b+ cells below a threshold value to determine a threshold value that indicates higher success of transfer implantation, and transferring the frozen embryo into the subject if the sample comprises a percent composition of gamma delta T cells above a threshold value and/or a percent composition of CD66b+ cells below a threshold value that indicates higher success of transfer implantation.

In some embodiments, the uterine cavity fluid sample contains cells.

In some embodiments, acquiring a uterine cavity fluid sample from the subject comprises passing a catheter inside the uterine cavity.

In some embodiments, performing the assay comprises performing a colorimetric assay. In some embodiments, the assay comprises a colorimetric assay configured to determine expression levels of CD66b in the sample.

In some embodiments, if the level of CD66b detected in a colorimetric ELISA assay is 1200 pg/ml or below, the transfer proceeds.

In some embodiments, the percent composition threshold value of gamma delta T Cells is about 24% or higher. In some embodiments, the percent composition threshold value of CD66b is about 39% or less.

In some embodiments, if the sample comprises a percent composition of gamma delta TCR cells below 24% or the sample comprises a percent composition of CD66b cells above 39%, the frozen embryo is not transferred into the subject.

In some embodiments, the subject is administered an anti-inflammatory therapeutic to reduce the level of immune cells in the uterine cavity fluid.

In some embodiments, the anti-inflammatory therapeutic comprises prednisone, progesterone or other anti-inflammatory agents.

Also disclosed is a system for frozen embryo transfer (FET) in a subject on day of embryo transfer, comprising a sample collection apparatus for collecting a uterine cavity fluid sample, and one or more assays to determine percent composition of immune cells within the sample collected from the subject on the day of transfer.

In various embodiments, the immune cells are selected from the group consisting of granulocytes, monocytes, or lymphocytes including neutrophils, eosinophils, basophils, mast cells, macrophages, histiocytes, dendritic cells, B cells, plasma cells, memory B cells, Killer T cells, Memory T cells, T helper cells, T regulatory cells, Natural Killer T cells, innate lymphoid cells, or Natural Killer cells.

In various embodiments, the immune cells express one or more cell surface markers selected from the group consisting of CD45, CD3, CD19, CD4, CD8, gamma delta (GD) TCR, CD25, CD127, CD66b, CD14, CD16, and CD56.

In some embodiments, the one or more assays is configured to determine the percent composition of immune cells in the sample. In some embodiments, the immune cells are gamma delta T cells and/or CD66b+ cells.

In some embodiments, the one or more assays comprise a colorimetric assay configured to determine expression levels of CD66b in the sample.

In some embodiments, the sample collection apparatus is a catheter or a needle.

Also disclosed is a kit for frozen embryo transfer comprising a sample collection apparatus for collecting a uterine cavity fluid sample and one or more assays to determine percent composition of immune cells within the sample collected from a subject on the day of transfer. In some embodiments, the kit further comprises instructions for us. In some embodiments, the sample collection apparatus is configured to collect a uterine cavity fluid sample that comprises immune cells from the uterine cavity of the subject and may include a catheter. In some embodiments, the one or more assays is an ELISA assay to measure cell surface markers such as CD66b. In some embodiments, the kit further comprises reagents for measuring immune cell surface markers. In some embodiments, the one or more assays may include a flow cytometry assay, and the kit may further comprise antibodies specific for one or more immune cell surface markers. In some embodiments, the one or more immune cell surface markers or antibodies to the one or more immune cell surface markers may be further described herein and may be useful in flow cytometry for detecting the cell surface markers.

It is understood that each feature or embodiment, or combination, described herein is a non-limiting, illustrative example of any of the aspects of the invention and, as such, is meant to be combinable with any other feature or embodiment, or combination, described herein. For example, where features are described with language such as “one embodiment”, “some embodiments”, “various embodiments”, “certain embodiments”, “further embodiment”, “specific exemplary embodiments”, and/or “another embodiment”, each of these types of embodiments is a non-limiting example of a feature that is intended to be combined with any other feature, or combination of features, described herein without having to list every possible combination.

Such features or combinations of features apply to any of the aspects of the invention. Where examples of values falling within ranges are disclosed, any of these examples are contemplated as possible endpoints of a range, any and all numeric values between such endpoints are contemplated, and any and all combinations of upper and lower endpoints are envisioned.

### BRIEF DESCRIPTION OF THE FIGURES

FIG. 1. depicts representative flow cytometry plots and immune subsets gating strategy for uterine cells including immune cells present in the uterine cavity fluid collected from females at the day of embryo transfer who underwent frozen embryo transfer (FET). Uterine cells were stained with live/dead kit first and then with the following surface markers: CD45, CD3, CD8, CD4, CD25, CD127, CD56, CD14, CD16, CD66b, and gamma delta TCR, acquired on Sony SP6800 Spectral Analyzer, and analyzed on FlowJo software.

FIG. 2A depicts a graph of viable uterine cell numbers collected from the uterine cavity fluid from the upper uterus or lower uterus of 24 females who underwent frozen embryo transfer (FET). Samples were collected from different locations in the uterus (upper and lower) on the day of embryo transfer, and pelleted cells were stained with live/dead kit and anti-CD45, acquired on Sony SP6800 Spectral Analyzer, and analyzed on FlowJo software.

FIG. 2B depicts a graph of the percentage of CD45+ cells in the uterine cavity fluid of the upper uterus or lower uterus.

FIG. 2C depicts a graph of the total number of CD45+ cells in the uterine cavity fluid of the upper uterus or lower uterus.

FIGS. 2D-2E depict pie charts of the percentage of CD45+ cells out of all viable cells in the upper uterus (FIG. 2D) and lower uterus (FIG. 2E). Values in pie charts represent mean±SD; (n=24+/−SD unpaired T-test (two-tailed) * p<0.05, ** p<0.01 and *** p<0.001).

FIGS. 3A-3J depict graphs of T, B, and NK cells in the uterine cavity fluid collected from different locations in the uterus (upper and lower) on the day of embryo transfer. Pelleted cells were sainted with live/dead kit and anti-CD45, anti-CD3, anti-CD8, anti-CD19, anti-CD25, anti-CD127, anti-CD56, anti-CD14, anti-CD16, anti-CD66b, and anti-gamma delta TCR, acquired on Sony flow cytometer, and analyzed on FlowJo software; (n=24 +/−SD unpaired T-test (two-tailed) * p<0.05, ** p<0.01 and *** p<0.001). FIGS. 3A-3B depict graphs of T cells (CD3) in the uterine cavity fluid collected from the upper uterus and lower uterus on the day of the embryo transfer. FIGS. 3C-3D depict graphs of B cells (CD19) in the uterine cavity fluid collected from the upper uterus and lower uterus on the day of the embryo transfer. FIGS. 3E-3F depict graphs of natural killer (NK) cells in the uterine cavity fluid collected from the upper uterus and lower uterus on the day of the embryo transfer. FIGS. 3G-3H depict graphs of CD56+ CD16+ cells in the uterine cavity fluid collected from the upper uterus and lower uterus on the day of the embryo transfer. FIGS. 31-3J depict graphs of CD56+ CD 16-cells in the uterine cavity fluid collected from the upper uterus and the lower uterus on the day of the embryo transfer.

FIGS. 4A-4H. Neutrophils, monocytes/macrophages and FcγRIII expressing cells in the uterine cavity fluid on the day of the embryo transfer. Uterine cavity fluid was collected from 24 females who underwent FET. Samples were collected from different locations in the uterus (upper and lower) on the day of embryo transfer, and pelleted cells were stained with live/dead kit and anti-CD45, anti-CD3, anti-CD8, anti-CD25, anti-CD127, anti-CD56, anti-CD14, anti-CD16, anti-CD66b, and anti-gamma delta TCR, acquired on Sony flow cytometer, and analyzed on FlowJo software. Values in pie charts represent mean±SD.; (n=24+/−SD unpaired T-test (two-tailed) * p<0.05, ** p<0.01 and *** p<0.001).

FIGS. 5A-5J. T cell (CD3+) subsets: GDT cells, CD8, CD4 T cells, and T reg cells in the uterine cavity fluid on the day of the embryo transfer. Uterine cavity fluid was collected from 24 females who underwent FET. Samples were collected from different locations in the uterus (upper and lower) on the day of embryo transfer, and pelleted cells were stained with live/dead kit and anti-CD45, anti-CD3, anti-CD8, anti-CD25, anti-CD127, anti-CD56, anti-CD14, anti-CD16, anti-CD66b, and anti-gamma delta TCR, acquired on Sony flow cytometer, and analyzed on FlowJo software. Values in pie charts represent mean±SD; (n=24+/−SD unpaired T-test (two-tailed) * p<0.05, ** p<0.01 and *** p<0.001).

FIG. 6. Cytokine analysis in the supernatant of uterine cells at the time of embryo transfer. Uterine cells were collected from 8 females who underwent FET. Pelleted cells (0.5-2×105 cells in 200 ul) were incubated in the culture medium (IMDM 10% FBS). Supernatants were collected after 24 h and BD™ CBA Human Th1/Th2/Th17 Cytokine Kit (BD Bioscience) was used to measure Interleukin-2 (IL-2), Interleukin-4 (IL-4), Interleukin-6 (IL-6), Interleukin-10 (IL-10), Tumor Necrosis Factor alpha (TNF-α), Interferon-gamma (IFN-γ) , and Interleukin-17A (IL-17A) protein levels in a single sample. Data represents mean±SD. Comparison of cell frequencies were measured by unpaired t-test (two-tailed) with Welch's correction (n=8). All statistical analysis was conducted at alpha 0.05 level using Graph Pad Prism versions 9.0 for Windows (GraphPad Software, San Diego, CA, graphpad.com).

FIG. 7A depicts a graph of the percent CD45+ cells in uterine cavity fluid acquired from the ongoing pregnancy group or the non-pregnancy group.

FIGS. 7B-7C depict pie charts of the percentage of CD45+ cells in all viable cells for the ongoing pregnancy group and for the non-pregnant group.

FIG. 7D depicts a graph of the total number of CD45+ cells for the ongoing pregnancy group and for the non-pregnant group.

FIG. 7E depicts a radar chart summarizing the percent distribution of live, CD45+ cells in the ongoing pregnancy group and non-pregnant group.

FIGS. 8A-81 depict graphs of the comparison of surface immune markers between the pregnant group (closed circle) and non-pregnant group (open circle). Surface immune markers include CD3 (FIG. 8A), CD19 (FIG. 8B), CD56 (FIG. 8C), CD14 (FIG. 8D), CD66b (FIG. 8E), CD4 (FIG. 8F), CD8 (FIG. 8G), TCR CD+ (FIG. 8H), and T_{regs} (FIG. 8I).

FIG. 8J depicts a radar chart summarizing the distribution of immune cells in ongoing pregnancy group (solid line) and non-pregnant group (dotted line).

FIG. 9A depicts a graph of a ROC curve presenting average values of the sensitivity for a percentage of TCR GD+ cells over all possible values of specificity.

FIG. 9B depicts a graph of a ROC curve presenting average values of the sensitivity for a percentage of CD66b+ cells over all possible values of specificity.

FIG. 10A depicts a standard curve of Human CD66b in lysed uterine cell samples from pregnant (P) and non-pregnant women (NP).

FIG. 10B depicts a graph of interpolated concentrations of CD66b in pregnant and non-pregnant lysed uterine cell samples.

FIG. 10C depicts a graph of the percentage of uterine leukocyte cells (CD45+) that express CD66b on their surface.

### DETAILED DESCRIPTION

Embryo implantation is a tightly regulated process with a very short period of uterine receptivity known as WOI [22]. Most studies are focusing on endometrial tissue to decipher molecular mechanisms responsible for successful implantation. However, tissue biopsy is an invasive procedure and can interfere with embryo implantation, especially on the day of transfer as it may disrupt the transfer. In addition, if endometrial tissue is obtained outside of the WOI, then molecular specifics of implantation site are misinterpreted.

One of the less explored areas is sampling of uterine cavity fluid. The composition of uterine cavity fluid (lipids, carbohydrates, amino acids, proteins, prostaglandins and miRNA) is changed during WOI [7-11]. Cellular composition of uterine cavity fluid has never been addressed before.

Immune cells are present in the pre/peri-implantation and pregnant endometrium tissue and immune cell activity correlates with recurrent implantation failure (RIF) [13-16]. Molecular mechanisms that control leukocyte migration across epithelial cells in the gut and lungs have been characterized in vivo [17-19]. Furthermore, dysregulated neutrophil recruitment across epithelial surfaces was linked to pathophysiology of numerous inflammatory diseases, such as inflammatory bowel disease (IBD) [20, 21].

The composition of immune cells in the uterine cavity may add to the complexity of uterine cavity fluid functions in facilitating pregnancy establishment. By examining the cells in the uterine cavity fluid at the time of the embryo transfer, more insight into the endometrial milieu present during transfer is gained, without any disruption of the endometrium at that time. Given the crucial role of immune cells in implantation and pregnancy, the role of the immune cell profile at the time of implantation can be used as a marker of receptivity. The presence or absence of such cells can be associated with clinical outcomes, such as live pregnancies.

### Methods for Frozen Embryo Transfer (FET)

Described here is a method for frozen embryo transfer (FET) that utilizes the analysis of uterine cavity immune cells at the time of embryo transfer for an indication of the success of transfer implantation.

Disclosed is a method for frozen embryo transfer (FET) in a subject. In some embodiments, the method comprises acquiring on the day of FET, a uterine cavity fluid sample from the subject, performing one or more assays on the uterine cavity fluid sample to measure immune cell composition on the sample, determining if the sample comprises a percent composition of immune cells above or below a threshold value of immune cell composition to provide a determined threshold value that indicates a higher success of transfer implantation, and transferring the froze embryo into the subject.

In some embodiments, acquiring the uterine cavity fluid sample from the subject comprises using a catheter to acquire a uterine cavity fluid sample from the uterine cavity of the subject. In some embodiments, acquiring the uterine cavity fluid sample from the subject includes passing the catheter inside the uterine cavity to acquire the uterine cavity fluid sample. In some embodiments, cells within the uterine cavity may be floating in a microfluidic compartment on top of epithelial cells that comprise the uterine cavity. In some embodiments, the microfluidic compartment comprises fluid that may be secreted by endometrial glands. In some embodiments, acquiring the uterine cavity fluid sample from the subject includes passing the catheter inside the uterine cavity to acquire a uterine cavity fluid sample of about 1 μL to about 15 μL including about 1 μL, about 2 μL, about 3 μL, about 4 μL, about 5 μL, about 6 μL, about 7 μL, about 8 μL, about 9 μL, about 10 μL, about 11 μL, about 12 μL, about 13 μL, about 14 μL, or about 15 μL. In some embodiments, acquiring the uterine cavity fluid sample from the subject comprises acquiring the uterine cavity fluid sample from the subject on the day of FET. In some embodiments, acquiring the uterine cavity fluid sample from the subject comprises acquiring a uterine cavity fluid sample from the subject that comprises a plurality of cells including immune cells. Advantageously, acquiring the uterine cavity fluid sample from the subject on the day of FET provides a strong indication of the likelihood of success of transfer implantation.

In some embodiments, acquiring the uterine cavity fluid sample from the subject comprises acquiring the uterine cavity fluid sample from the subject about 24 hours before FET, about 23 hours before FET, about 22 hours before FET, about 21 hours before FET, about 20 hours before FET, about 19 hours before FET, about 18 hours before FET, about 17 hours before FET, about 16 hours before FET, about 15 hours before FET, about 14 hours before FET, about 13 hours before FET, about 12 hours before FET, about 11 hours before FET, about 10 hours before FET, about 9 hours before FET, about 8 hours before FET, about 7 hours before FET, about 6 hours before FET, about 5 hours before FET, about 4 hours before FET, about 3 hours before FET, about 2 hours before FET, or about 1 hour before FET. In some embodiments, acquiring the uterine cavity fluid sample from the subject comprises acquiring the uterine cavity fluid sample from the subject about 60 minutes before FET, about 45 minutes before FET, about 30 minutes before FET, about 15 minutes before FET or about 5 minutes before FET.

In some embodiments, acquiring the uterine cavity fluid sample may comprise acquiring multiple uterine cavity fluid samples from different locations of the uterine cavity. In some embodiments, acquiring the uterine cavity fluid sample may comprise passing the catheter within different locations of the uterine cavity to acquire different uterine cavity fluid samples. For example, in some embodiments, acquiring the uterine cavity fluid sample may comprise acquiring a sample from the upper uterus and/or the lower uterus. In some embodiments, the multiple uterine cavity fluid samples from different locations of the uterine cavity may be combined into a single sample that may be processed and used for analysis.

In some embodiments, the method further comprises performing one or more assays on the uterine cavity fluid sample to measure the immune cell composition of the sample. In some embodiments, performing one or more assays on the uterine cavity fluid sample may comprise further processing the uterine cavity fluid sample including using a live/dead detection kit on the sample to separate the live immune cells from the dead immune cells before further performing one or more assays on the uterine cavity fluid sample.

In some embodiments, the one or more assays may be configured to measure any one or more immune cells including, but not limited to: granulocytes, monocytes, or lymphocytes including neutrophiles, eosinophils, basophils, mast cells, macrophages, histiocytes, dendritic cells, B cells, plasma cells, memory B cells, Killer T cells, Memory T cells, T helper cells, T regulatory cells, Natural Killer T cells, innate lymphoid cells, or Natural Killer cells. In some embodiments, the immune cells may comprise gamma delta T cells and/or CD66b+ cells. In some embodiments, the one or more assays may comprise colorimetric assays. In some embodiments, the one or more assays may comprise a colorimetric ELISA assay. In some embodiments, the colorimetric ELISA assay may be specific for CD66b.

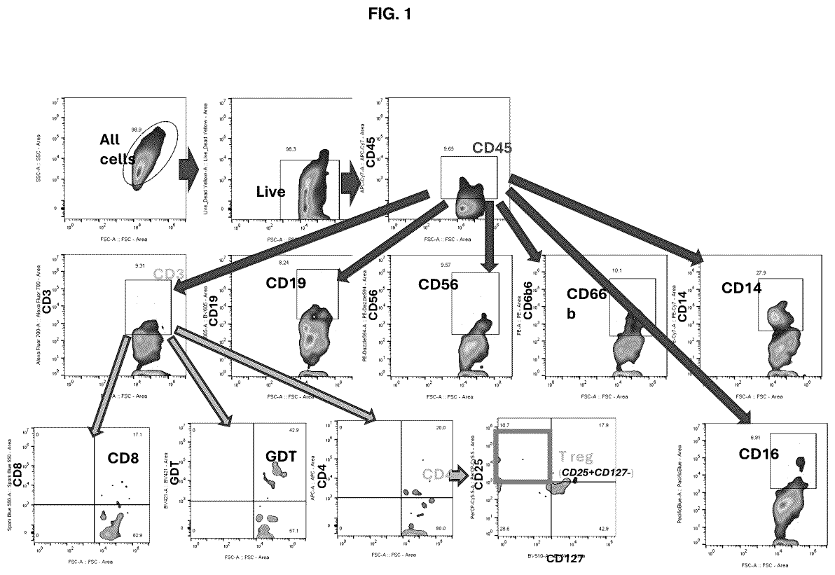

In some embodiments, the one or more assays may comprise contacting the uterine cavity fluid sample with fluorescently conjugated antibodies to one or more cell surface markers on the cell surface of the immune cells and performing flow cytometry analysis. In some embodiments, the flow cytometry analysis may comprise performing the gating strategy depicted in FIG. 1.

In various embodiments, the immune cells express one or more cell surface markers selected from the group consisting of CD45, CD3, CD19, CD4, CD8, gamma delta (GD) TCR, CD25, CD127, CD66b, CD14, CD16, and CD56.

In some embodiments, the method further comprises determining if the sample comprises a percent composition of immune cells above or below a threshold value of immune cell composition. In some embodiments, the percent composition of immune cells in the uterine cavity fluid sample may provide a determined threshold value that indicates a higher or lower success of transfer implantation. In some embodiments, determining if the sample comprises a percent composition of immune cells above or below a threshold value of immune cell composition may include using a ROC curve analysis to determine the determined threshold value of the percent composition of immune cells that indicates a higher or lower success of transfer implantation.

In some embodiments, the percent composition of immune cells in the uterine cavity fluid sample may comprise the percent composition threshold of gamma delta T cells. In some embodiments, the percent composition of immune cells in the uterine cavity fluid sample may comprise the percent composition threshold of CD66b+ cells. In some embodiments, the percent composition of immune cells in the uterine cavity fluid sample may comprise the percent composition threshold of gamma delta T cells or CD66b+ cells.

In some embodiments, wherein the immune cells in the uterine cavity fluid sample comprise gamma delta T cells, the percent composition threshold value may be about 5%, about 10%, about 15%, about 16%, about 17%, about 18%, about 19%, about 20%, about 21%, about 22%, about 23%, about 24%, about 25%, about 26%, about 27%, about 28%, about 29%, or about 30%. In some embodiments, the percent composition threshold value for gamma delta T cells may be about 24%, wherein a percent composition threshold value above 24% indicates a high success of transfer implantation, and a percent composition threshold value below 24% indicates lower success of transfer implantation.

In some embodiments, wherein the immune cells in the uterine cavity fluid sample comprise CD66b+ cells, the percent composition threshold value may be about 20%, about 25%, about 26%, about 27%, about 28%, about 29%, about 30%, about 31%, about 32%, about 33%, about 34%, about 35%, about 36%, about 37%, about 38%, about 39%, about 40%, about 41%, about 42%, about 43%, about 44%, about 45%, or about 50%. In some embodiments, the percent composition threshold value for CD66b+ cells may be about 39%, wherein a percent composition threshold value below 39% indicates a high success of transfer implantation and a percent composition threshold value below 39% indicates a lower success of transfer implantation.

In some embodiments, the method further includes transferring the frozen embryo into the subject. In some embodiments, transferring the frozen embryo into the subject comprises transferring the frozen embryo into the subject if the sample comprises a percent composition of immune cells above a determined threshold value. In some embodiments, transferring the frozen embryo into the subject comprises transferring the frozen embryo into the subject if the sample comprises a percent composition of immune cells below a threshold value that indicates high success of transfer implantation.

In some embodiments, transferring the frozen embryo into the subject comprises transferring the embryo into the subject if the percent composition of CD66+ within the uterine cavity fluid sample is below the percent composition threshold value of 39%.

In some embodiments, transferring the frozen embryo into the subject comprises transferring the embryo into the subject if the percent composition of gamma delta T cells within the uterine cavity fluid sample is above the percent composition threshold value of 24%.

In some embodiments, transferring the frozen embryo into the subject comprises transferring the embryo into the subject if the percent composition of CD66b+ within the uterine cavity fluid sample is below the percent composition threshold value of 39% and the percent composition of gamma delta T cells within the uterine cavity fluid sample is above the percent composition threshold value of 24%.

In some embodiments, if the percent composition of CD66+ within the uterine cavity fluid sample is above the percent composition threshold value of 39% and/or the percent composition of gamma delta T cells within the uterine cavity fluid sample is below the percent composition threshold value of 24%, the frozen embryo will not be transferred into the subject. In some embodiments, if the level of CD66b detected in a colorimetric ELISA assay is 1200 pg/ml or below, the transfer proceeds. In some embodiments, if the level of CD66b detected in a colorimetric ELISA assay is 1201 pg/ml or above the transfer does not proceed.

In some embodiments, instead of transferring the frozen embryo into the subject, the subject may be administered an anti-inflammatory therapeutic to reduce the level of immune cells in the uterine cavity fluid. In some embodiments, the anti-inflammatory therapeutic may comprise prednisone, progesterone, or other anti-inflammatory agents including, for example, antibiotics, and nonselective NSAIDS, and selective COX-2 inhibiting NSAIDS.

### System for Frozen Embryo Transfer (FET) in a Subject on the Day of Embryo Transfer

Also disclosed is a system for frozen embryo transfer in a subject on the day of embryo transfer. In some embodiments, the system comprises a sample collection apparatus for collecting a uterine cavity fluid sample and one or more assays to determine percent composition of immune cells within the sample collected from the subject on the day of transfer. In some embodiments, the sample collection apparatus is configured to collect a uterine cavity fluid sample that comprises immune cells from the uterine cavity of the subject. In some embodiments, the sample collection apparatus is a catheter.

In some embodiments, the one or more assays may generate a determined threshold value that indicates a higher or lower success of transfer implantation. In some embodiments, the one or more assays to determine the percent composition of immune cells within the uterine cavity fluid sample may comprise a colorimetric assay. In some embodiments, the one or more assays may comprise an ELISA assay. In some embodiments, the one or more assays may comprise a flow cytometry assay. In some embodiments, the one or more assays may comprise contacting the uterine cavity fluid sample with one or more fluorescently conjugated antibodies to one or more cell surface markers on the cell surface of the immune cells. In some embodiments, the one or more assays may be configured to determine the percent composition of gamma delta T cells and/or CD66b+ cells within the uterine cavity fluid sample. In some embodiments, the colorimetric assay may be configured to determine the presence or expression levels of CD66b+ cells in the sample.

In various embodiments, the sample collection apparatus for collecting a uterine cavity fluid sample is a catheter or needle.

### Kits

Also provided is a kit for frozen embryo transfer comprising a sample collection apparatus for collecting a uterine cavity fluid sample and one or more assays to determine percent composition of immune cells within the sample collected from the subject on the day of transfer, optionally the kit comprises instructions for use. In some embodiments, the sample collection apparatus is configured to collect a uterine cavity fluid sample that comprises immune cells from the uterine cavity of the subject. In some embodiments, the sample collection apparatus is a catheter. In some embodiments, the one or more assays is an ELISA assay to measure cell surface markers such as CD66b and the kit comprises reagents for measuring immune cell surface markers. In some embodiments, the one or more assays is a flow cytometry assay and the kit comprises antibodies specific for one or more immune cell surface markers as described herein useful in flow cytometry for detecting the cell surface marker.

### Subjects

In some embodiments, the subject is a female adult. In some embodiments, the female adult is of any suitable age. In some embodiments, the female adult may be at least 18 years of age and no more than 60 years of age. In some embodiments, the female adult may be at least about 30 years of age, at least about 31 years of age, at least about 32 years of age, at least about 33 years of age, at least about 34 years of age, at least about 35 years of age, at least about 36 years of age, at least about 37 years of age, at least about 38 years of age, at least about 39 years of age, at least about 40 years of age, at least about 41 years of age, at least about 42 years of age, at least about 43 years of age, at least about 44 years of age, or at least about 45 years of age.

In some embodiments, the subject may be currently receiving the administration of hormones. In some embodiments, the subject may be receiving the administration of estrogen and/or progesterone. In some embodiments, the subject may be receiving a treatment regimen of hormones. For example, in some embodiments, the subject may be receiving estrogen for about 1 day, about 2 days, about 3 days, about 4 days, about 5 days, about 6 days, about 7 days, about 8 days, about 9 days, about 10 days, about 11 days, about 12 days, about 13 days, about 14 days, about 15 days, about 16 days, about 17 days, about 18 days, about 19 days, about 20 days, or about 21 days.

In some embodiments, the subject may be receiving progesterone for about 1 day, about 2 days, about 3 days, about 4 days, about 5 days, about 6 days, about 7 days, about 8 days, about 9 days, about 10 days, about 11 days, about 12 days, about 13 days, about 14 days, about 15 days, about 16 days, about 17 days, about 18 days, about 19 days, about 20 days, or about 21 days.

In some embodiments, the subject may have an endometrial thickness at the beginning of progesterone administration of about 5 mm, about 6 mm, about 7 mm, about 8 mm, about 9 mm, about 10 mm, about 11 mm, about 12 mm, about 13 mm, about 14 mm, about 15 mm, about 16 mm, about 17 mm, about 18 mm, about 19 mm, or about 20 mm.

### Frozen Embryo Transfer

In some embodiments, a frozen embryo is transferred to the uterus of the subject. In some embodiments, a single frozen embryo is transferred to the subject. In some embodiments, standard transfer methods are used to transfer the frozen embryo to the subject.

### EXAMPLES

### Example 1

The study described herein is the first to analyze the composition of immune cells in the uterine cavity to investigate uterine fluid functions in facilitating pregnancy establishment.

### Materials and Methods

Study design, size, duration: The study was designed as a prospective observational cohort study between May 2021 and December 2022 at a single academic-based fertility center. All patients underwent at least one IVF cycle and have at least one frozen embryo available for transfer.

Study Participants: The study cohort included a total of 24 women with infertility. The characteristics of the subjects can be seen in Table 1. The average age at the time of oocyte retrieval was 34.75 years old and the body mass index was 29.33 kg/m². The mean duration of endometrial preparation with estrogen was 13 days and progesterone was added for 6 days prior to transfer. The average endometrial thickness prior to embryo transfer was 10.13 mm. At the time of oocytes fertilization, 23 patients underwent IVF with intracytoplasmic sperm injection (ICSI), while one patient underwent conventional IVF, i.e. without ICSI. Only single embryo transfer was performed in all cases. Patients eligible to participate in the study were those of age 18 to 55 years old who underwent a cycle of IVF which resulted in at least one frozen embryo available for transfer regardless of indication for IVF.

A total of 24 participants were recruited for the study after providing written informed consent. Endometrial preparation was performed per the institution's protocol of hormone replacement treatment. Patients were started on oral estradiol on day 3 of the cycle and increased in an incremental fashion with the addition of transdermal estradiol. Luteal phase support was then provided with intramuscular progesterone. Patients were monitored with transvaginal ultrasound (US). Patients then presented for embryo transfer after 6 days of progesterone administration.

### Uterine Cavity Fluid Collection and Cell Preparation

Prior to transfer of the embryo, a trial transfer catheter (Rocketmedical, U.K.) was used to determine the trajectory of the cervix and is placed into the lower uterine segment under transabdominal US guidance. The trial catheter was then removed and prior to discarding, the tip is rinsed in Iscove's Modified Dulbecco's Medium (IMDM) with 10% fetal bovine serum (FBS) (both reagents from Gibco, Billings, Montana, U.S.), which consisted of the first specimen, labeled as “lower.” Subsequently, the transfer catheter was loaded with the embryo, and the embryo was then placed under US guidance into the upper uterus approximately 1-2 cm from the fundus (Rocketmedical, U.K.). The catheter was then removed and provided to the embryologist to verify that the catheter does not contain the embryo. The tip of this catheter was then rinsed in a separate aliquot of IMDM with 10% FBS, which consists of the second specimen, labeled as “upper.” The specimens were then put on ice and then transferred within 30 min to the laboratory for analysis.

### Flow Cytometry

Uterine samples including lower and upper uterine samples were spun in refrigerated centrifuge and pelleted cells were stained first with Yellow Live/Dead detection kit (ThermoFisher Scientific, Waltham, Massachusetts, U.S.) followed by surface marker staining. Fluorochrome conjugated antibodies against the following surface markers were used in this study: CD45, CD3, CD19, CD4, CD8, gamma delta (GD) TCR, CD25, CD127, CD66b, CD14, CD16 and CD56. The following antibodies were purchased from BioLegend (San Diego, California, U.S.): APC/Cy7 anti-human CD45/Clone HI30, Brilliant Violet 605 anti-human CD19/Clone HIB19, APC anti-human CD4/Clone GK1.5, Spark Blue 550 anti-human CD8/Clone 53-6.7, Brilliant Violet 421 anti-human gamma delta TCR/Clone B1, PercP/Cyanine5.5 anti-human CD25/Clone BC96, Brilliant Violet 510 anti-human CD127/Clone A019D5, PE anti-human CD66b/Clone HCD56, PE/Cy7 anti-human CD14/CloneM5E2, Pacific Blue anti-human CD16/Clone 3G8, PE/Dazzle 594 anti-human CD56/Clone HCD56, or from BD Pharmingen (San Diego, California, US): Alexa Fluor 700 anti-human CD3/Clone SP34-2, and acquired on Sony SP6800 Spectral Analyzer.

Analysis was performed using FlowJo 10 software (Tree Star). Gating strategy is shown (FIG. 1). First, cells were gated on live cells and then on CD45+ and main cell subsets (CD3+, CD19+, CD56+, CD66b+, CD14+, and CD16+ cells) were analyzed within the CD45+ T cells. Progressive gating on CD3+ T cell subsets was used to determine T cell subsets: CD8+, CD4+ and gamma delta T (GDT+) cells. T regulatory cells were determined as CD4+CD25+CD127-cells within gated CD4+ cells.

### Cytokine Analysis

Uterine cells were collected from 8 females who underwent FET. Cells (0.5-2×10⁵ cells in 200 ul) were incubated in the culture medium (IMDM 10% FBS) and supernatants were collected after 24 h. BD Cytometric bead array (CBA) assay, BD™ CBA Human Th1/Th2/Th17 Cytokine Kit (BD Biosciences, Franklin Lakes, NJ, U.S.), was used to measure cytokines in cell supernatants: Interleukin-2 (IL-2), Interleukin-4 (IL-4), Interleukin-6 (IL-6), Interleukin-10 (IL-10), Tumor Necrosis Factor alpha (TNF-α), Interferon-γ (IFN-γ), and Interleukin-17A (IL-17A). The kit performance has been optimized for analysis of physiologically relevant concentrations (pg/mL levels) of specific cytokine proteins in tissue culture following kit protocol.

### Statistical Analysis

Differences in frequency and total numbers between the two groups (upper and lower uterus) were analyzed with unpaired T-test (two-tailed) and Welch's correction was applied with unpaired T test, when P value of the F test to compare variances were ≤0.05. Data were presented as mean±standard deviation in the text and in the figures. All statistical analysis was conducted at alpha 0.05 level using GraphPad Prism versions 9.0 for Windows (GraphPad Software, San Diego, CA, graphpad.com).

### Hematopoietic Cells (CD45+ Cells) Are Present in the Uterine Cavity Fluid Sample Collected at the Time of Frozen Embryo Transfer (FET)

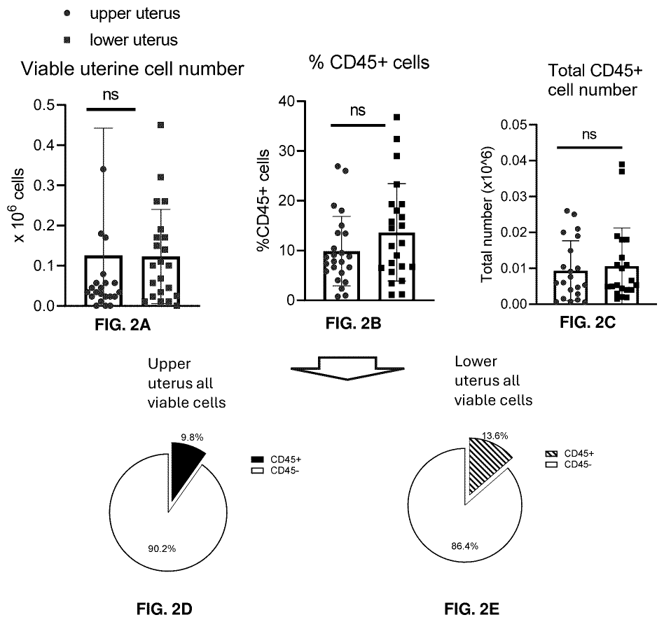

Uterine cavity fluid was collected from 24 females who underwent FET at two different locations in the uterus: upper and lower uterus, as described in Material and Methods on the day of FET. The viability of the uterine cavity fluid cells was first analyzed by staining cells with the Yellow Viability dye (FIG. 1). The viability of all collected samples was between 48-85%. We found 0.125×10⁶ live cells+/−0.32 SD in the samples collected in the upper uterus, while in the samples collected in the lower uterus 0.123×10⁶ live cells+/−0.12 SD were found (FIG. 2A). To determine frequency of hematopoietic immune cells within the collected uterine cavity fluid cells, cells were stained with anti-CD45 antibody. In the upper uterus 9.88%+/−6.98 SD of all analyzed viable cells were CD45+, and 13.67%+/−9.79 SD in the lower uterus were CD45+ (seen in FIG. 2B). Among total viable cells, there was no significant difference in the percentage (9.88%+/−6.98 SD and 13.67%+/−9.79 SD, p=0.198) (FIG. 2C 0 and number (0.0093×10⁶+/×1×10⁻² SD and 0.0100×10⁶+/−1×10⁻² SD) of CD45+ cells between the upper and lower uterus (FIGS. 2D-2E).

### T, B and NK Cells in the Uterine Cavity Fluid Sample

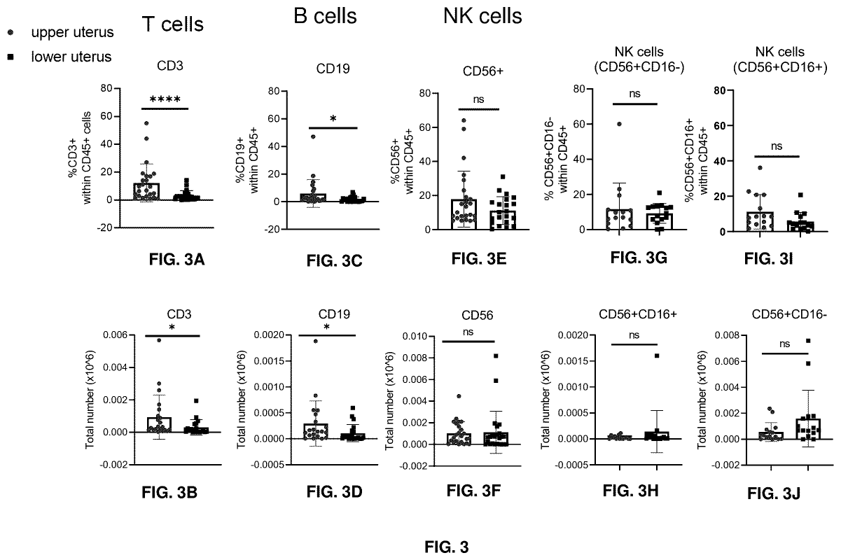

In order to determine major immune cell subsets in the uterine cavity fluid samples collected on the day of frozen embryo transfer from different locations in the uterus, cells were spun down and stained with live/dead kit, followed by antibodies for cell-surface markers CD45, CD3, CD8, CD25, CD127, CD56, CD14, CD16, CD66b, and gamma delta (GD) TCR. T, B and natural killer (NK) cell subsets were first analyzed (FIGS. 3A-3J). After gating on all live, CD45+ cells, CD3+ T cells were analyzed (FIGS. 3A-3B); significantly higher frequency and total number of CD3+ (p=0.006 and p=0.016) were found in the samples collected from upper uterus (12.20%+/−13.60 SD and 0.0009×10⁶+/−1.3×10⁻³ SD) compared to lower uterus (3.09%+/−3.60 SD and 0.0003×10⁶+/−4×10⁻⁴ SD).

Significantly higher frequency and total number of CD19+ cells (B cells) were identified in samples collected from upper uterus (5.80%+/−9.90 SD and 0.00029×10⁶+/−4×10⁻⁴ SD) compared to lower uterus (1.60%+/−1.80 SD and 0.00010×10⁶+/−1×10⁻⁴ SD) (FIGS. 3C-3D). Frequency and total number of CD56+ cells (NK cells) were similar in upper (17.9%+/−16.40 SD and 0.0010×10⁶+/−1×10⁻³ SD) and lower uterus (11.20%+/−8.10 SD and 0.0011×10⁶+/−1×10⁻³ SD) (FIGS. 3E-3F).

### Neutrophils, Monocytes/Macrophages and FcγRIII Expressing Cells in the Uterine Cavity Fluid Sample

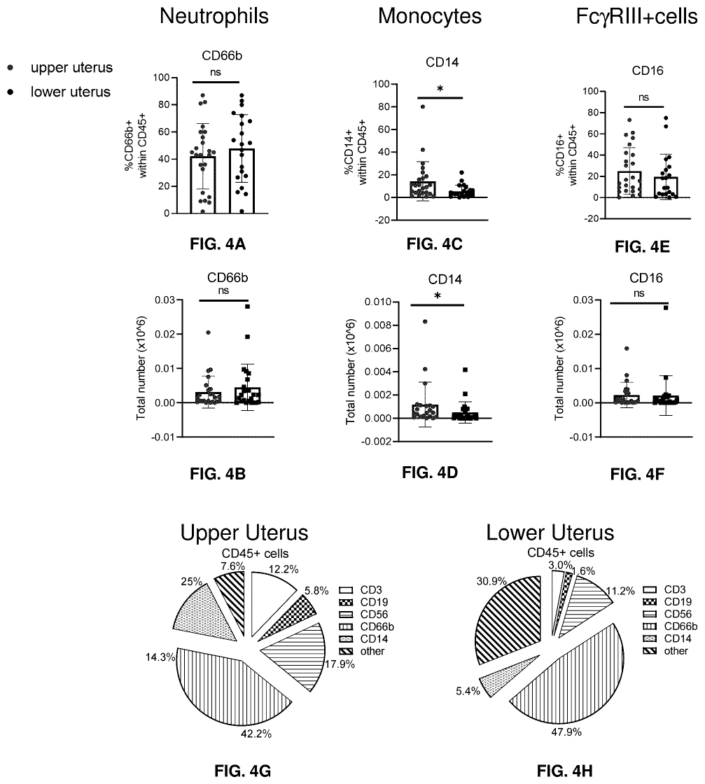

The frequency of neutrophils, monocyte/macrophages and FcγRIII expressing cells in the uterine cavity fluid were determined by gating on live, CD45+ cells and analyzing expression of CD66b, CD14 and CD16. CD16 (FcγRIII) is found on the cell surface of multiple cell types: NK cells, neutrophils, monocytes, macrophages, and certain T cells. CD16+ cells included all of the above cell types. A statistically significant difference was found in the frequency (p=0.018) and number (p=0.021) of monocyte/macrophages (CD14+ cells) in the upper uterus (14.30%+/−17.30 SD and 0.00029×10⁶+/−4×10⁻⁴ SD) compared to lower uterus (5.50%+/−5.40 SD and 0.0001×10⁶+/−1×10⁻⁴ SD) (FIGS. 4C-4D). However, there was no statistically significant difference in the frequency (p=0.401) or number (p=0.311) of neutrophils (CD66b+) (FIGS. 4A-4B) and FcγRIII+ (CD16+) cells (frequency p=0.2541 and number p=0.3114) between upper and lower uterus (FIGS. 4E-4F). FIGS. 4G and 4H depict pie charts of the composition of immune cells within the upper uterus and the lower uterus.

### T Cell Subsets: Gamma Delta (GD) T Cells, CD8, CD4 T Cells, and T Reg Cells in the Uterine Cavity Fluid Sample

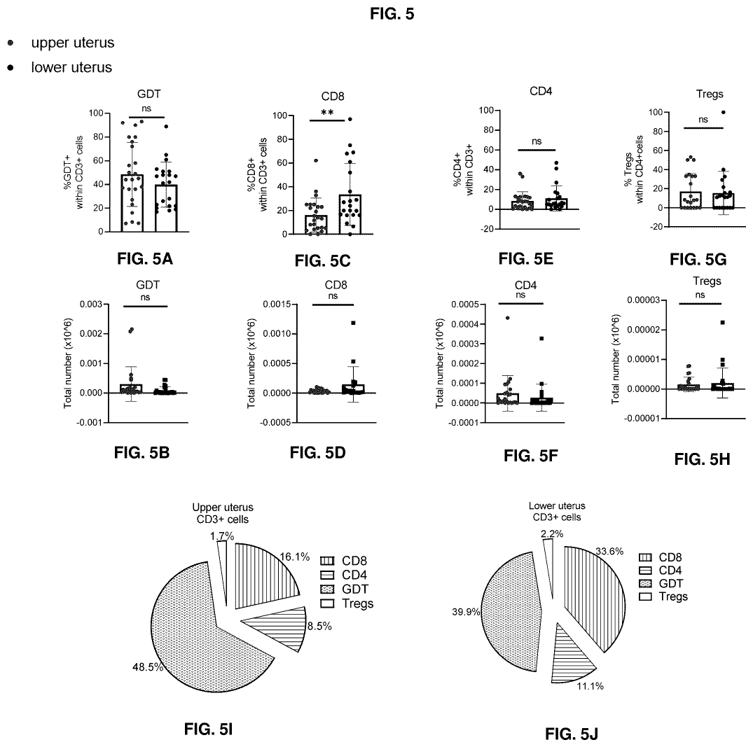

The frequency of T cell subsets in uterine cavity fluid was determined by gating on live, CD45+CD3+ cells and analyzing expression of CD8, gamma delta (GD) TCR, CD25 and CD127 expression. T regulatory cells (Tregs) were determined as CD3+CD4+GD TCR-CD25+CD127−. Within all T cells (CD3+ cells), GD T cells were the major population of T cells in both upper (48.50%+/−27.07 SD) and lower (39.90%+/−19.04 SD) uterus (FIGS. 5A-5B). CD8+ T cells were significantly higher (p=0.009) in the lower uterus (33.60%+/−26.15 SD) when compared to the upper uterus (16.10%+/−14.20 SD) (FIGS. 5C-5D). There was no statistically significant difference in the expression of CD4+ T cells (p=0.630) and T regulatory cells (p=0.770) between the upper and lower uterus (FIGS. 5E-5H). FIGS. 51-5J depict pie charts of the composition of T cell subsets within the upper uterus and the lower uterus.

Uterine Cavity Fluid Cells Ex-Vivo Produce TNF-α, IFN-γ, IL-6, IL-4, and IL-2

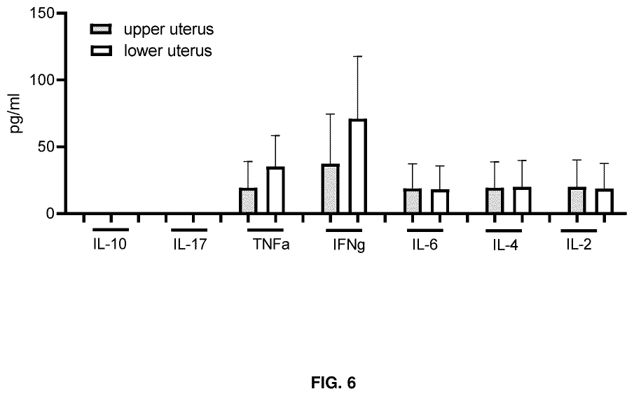

The production of IL-10, IL-17, TNF-α, IFN-γ, IL-6, IL-4 and IL-2 in the supernatant of the uterine cells was measured after 24 h culture (FIG. 6). Uterine cells from both compartments, upper and lower uterus, produce TNF-α, IFN-γ, IL-6, IL-4 and IL-2. Uterine cells produced between 19.40 pg/ml+/−55 SD and 35.30 pg/ml+/−65 SD of TNF-α and 37.20 pg+/−105 SD and 71.00 pg/ml+/−131 SD of IFN-γ from lower and upper uterine cavity fluid respectively (FIG. 6). Production of IL-6, IL-4 and IL-2 was between 18.00 and 20.00 pg/ml. There were no significant differences in the cytokine production between upper and lower uterus.

### Discussion

This disclosure provides a unique method of analyzing uterine cavity fluid immune cells at the time of the embryo implantation, providing a potential new diagnostic tool to measure cellular markers of endometrial receptivity.

Embryo implantation is a tightly regulated process with a very short period of uterine receptivity known as WOI [22]. Most of the studies are focusing on endometrial tissue to decipher molecular mechanisms responsible for successful implantation. However, tissue biopsy is an invasive procedure and can interfere with embryo implantation. In addition, if endometrial tissue is obtained outside of the WOI, then molecular specifics of implantation site are misinterpreted. In this study we propose that analysis of uterine cavity immune cells at the time of embryo transfer represents an alternative method to study endometrial receptivity. Flow cytometry was utilized to define the hematopoietic cells that are present in the uterine cavity at the time of embryo transfer.

For the first time, the study described herein showed that uterine cavity fluid contains not only viable endometrial (CD45 negative) cells but also about 10% of all viable hematopoietic (CD45 positive) cells (FIGS. 2B-2C). Immune cells, together with cytokines/chemokines and other soluble factors and hormones, are necessary components of successful implantation. Perfectly coordinated interplay between immune cells, hormones, and cytokines is crucial during peri-implantation [23]. The most prevalent cell subtype in the early maternal-fetal interface are uterine NK cells (up to 70% of all decidual leukocytes). Uterine NK cells are also the most common leukocytes in the non-pregnant endometrial tissue, with increasing quantity after ovulation [24]. They have a significant impact on maternal spiral artery remodeling and angiogenesis [25]. Studies have demonstrated a role of uterine NK cells in pathologies such as recurrent pregnancy loss, RIF, and infertility overall [13, 24, 26-29]. However, it is not known how uterine NK cells gain their way to the lumen of the uterine cavity and what is their impact on the implantation. The study described herein, for the first time, assesses the cellular composition of uterine fluid, revealing that immune cells within the uterine cavity, not solely confined to the endometrium, may play a crucial role in supporting implantation of the blastocyst. By incorporating the CD16 marker into the antibody panel, the study differentiated between cell subsets expressing CD16 (FIGS. 3E-3J), which have distinct functions. For instance, CD56+ CD16-positive cells, predominantly found in peripheral blood, exhibit higher cytotoxicity compared to CD56+ cells that are CD16-negative. The latter produce elevated levels of cytokines such as IFN-γ, IL-8, and VEGF, and constitute a predominant NK cell population in the decidua. In future studies, we aim to include additional markers of activation (CD25, CD69), memory differentiation (CD44, CD62L), and cytotoxic potential (perforin, granzyme, CD107a) to provide a more comprehensive understanding of the functional status of uterine cavity fluid NK cells regarding the pregnancy outcome.

The study revealed that T and B cells are present in the uterine cavity at the time of embryo transfer (FIGS. 3A and 3C). T and B cells have been implicated in the process of embryo implantation [30, 31]. Both cell types exhibit regulatory properties and protect the fetus from infection but can also lead to complications in the case of produced autoantibodies [32, 33]. CD3+ cells constituted 3% (lower) and 12.2% (upper) of all live CD45+ cells, which is in line with reported data about first trimester pregnancy T cells which constitute approximately 5-20% of total decidual leukocytes (FIG. 3B). The difference in the frequency of CD3+ T cells between upper and lower is statistically significant (p<0.05).

Furthermore, all three T cell subsets (CD8, CD4 and GDT cells) were assessed, and it was found that around 48% of all T cells in the upper uterus and 39.9% in the lower uterus are composed of GDT cells, representing the major T cell subset in the uterine cavity (FIG. 5A). This is in line with reported data that decidual GDT cells account for the major T cell subset in human decidua [34, 35]. Decidual GDT cells participate in maintenance of pregnancy by recognizing alloantigen without MHC restriction, producing cytokines and linking the innate and adaptive immune responses as a bridge [36]. Recently, it was reported that decidual stromal cell-derived RANKL promotes the polarization of decidual GDT cells into Foxp3+ regulatory GDT cells and elevates TGF-β1 production by activating NF-κB pathway [37]. Regulatory T cells increase in quantity throughout pregnancy and are considered vital for implantation and the early pregnancy period [30, 38]. Various studies have shown alterations in Tregs in the case of idiopathic infertility, RIF, and chronic endometritis [15, 24, 39]. Interestingly, of all T cell subsets characterized in the study, Tregs frequencies and numbers were the lowest (1.7% in the upper and 2.2% in the lower uterus) (FIG. 5H). It may be that decidual Tregs at the time of embryo transfer are not equipped with the surface markers that will allow them to migrate from the tissue to the uterine lumen or that the environment within the uterine cavity does not support migration of Tregs.

CD8 T cells are necessary to establish a healthy pregnancy via optimal immune regulation to provide protection again infections while avoiding a harmful response to allogeneic fetal cells [40]. Altered presence of CD8 and CD4 T cells have been associated with pregnancy outcomes after fresh and frozen embryo transfer, idiopathic infertility, and RIF [14, 26, 27]. A 3:1 ratio of CD8 to CD4 T cells was identified in the lower, and a 2:1 ratio in the upper uterus (FIGS. 51-5J), which is in line with the reported data about first trimester human decidual CD8 cells that constitute approximately 45% of total T cells [41-43]. Within the nonpregnant human uterus, CD8+ T cells have cytotoxic capabilities with fluctuating activity throughout the menstrual cycle [44]. Significantly higher frequency of CD8 T cells within the T cells of lower uterus (FIG. 5C) could be explained by their potential cytotoxic role in the pathogen clearance, assuming that the lower uterus harbors more diverse microbiome than the upper uterus. On the other hand, decidual CD8+ T cells, especially the effector memory (EM) subset, often display an exhausted profile and tissue specific features, which in homeostasis favor an intense production of cytokines [41, 45] rather than cytolytic programs.

Neutrophils (CD66b+ cells) function as short-lived effector cells, and in general, they do not reside in tissues but are recruited from the circulation to sites of inflammation or infection [23, 46]. Similar to other decidual cells (decidual NK cells and macrophages) decidual neutrophils adopt a unique phenotype that is different from the phenotype of systemic (blood) neutrophils with decidual neutrophils expressing high levels of neutrophil activation markers and angiogenesis-related proteins such as VEGF, Arginase-1 (ARG-1), and chemokine ligand-2 (CCL2) [47], suggesting that decidual neutrophils display angiogenic properties that are vital for successful implantation. Furthermore, several mammalian studies have noted an association with efficient blastocyst implantation and neutrophils [48-50]. Opposite to this positive function, excessive number of neutrophils at maternal-fetal interface leads to enhanced oxidative damage, having a deleterious effect on pregnancy and early fetal death [51]. The study described herein revealed very high frequency of neutrophils within the live, uterine cavity fluid leukocytes (47.9% in lower and 42.2% in upper uterus) (FIG. 4A) which is consistent with the overall leukocytes' recruitment in the pre-ovulatory phase and their pro-inflammatory activity that could further facilitate receptive endometrium.

Finally, monocytes/macrophages (CD14+ cells) consist of the second largest component of leukocytes in the decidua with increasing infiltration as the pregnancy progresses. Decidual macrophages were found to have activated phenotype and therefore able to present antigens to T cells. They have also been described to have M2, immunosuppressive phenotype that secrete TGF-β and IL-10 [53, 54]. In this study, significantly decreased frequency and number of CD14+ cells was observed in the lower uterus when compared to upper uterus (FIG. 4C) which could be the result of higher density of decidual macrophages in upper uterus, predisposed placentation sites compared to lower uterus [55, 56].

It is well accepted that a pro-inflammatory environment is required for successful blastocyst implantation, however inability to regulate excessive inflammatory responses at the maternal-fetal interface will result in implantation failure. Khadem et al., performed a study in which they explored the association of IL-1 and TNF-α concentration in endometrial secretions to endometrial receptivity and IVF outcomes, and they determined that increased levels can be associated with improved outcomes [5]. Assuming that leukocytes in the uterine cavity could be implicated in the cytokine production, we measured cytokines secreted from uterine cavity fluid cells (upper and lower uterus) after 24 h in vitro culture. It was found that uterine cells produce TNFα (19.40 and 35.30 pg/ml) and IFN-γ (37.20 and 71.00 pg/ml), and production of IL-6, IL-4 and IL-2 was less than 20.00 pg/ml. Since IFN-γ is primarily secreted by activated T cells and NK cells, T cells and NK cells within the uterine cavity can mediate antiviral/antibacterial immunity, promote macrophage activation and enhance their antigen presentation, coordinate lymphocyte-epithelium and endothelium interaction, and overall regulate Th1/Th2 balance [57, 58]. TNF-α is a Th1 cytokine and is secreted from immune cells as well as from the endometrial stromal cells to initiate

inflammatory action that may reach its optimal efficacy at the WOI [60]. Furthermore, in a prospective study, women who ultimately miscarried exhibited a significantly lower IL-10/TNF-α ratio circulating in maternal blood in the first trimester preceding the loss compared to women with ongoing pregnancies, supporting the possible deleterious effects of an exaggerated proinflammatory milieu [61].

In summary, the study described herein confirmed the presence of different immune cell types within the uterine fluid, verifying the utility of this method for future assessment of leukocyte trans-epithelial migration and their association with implantation success or failure.

### Example 2

An additional study was undertaken to determine the effects of immune cells in the uterine cavity fluid on the success of embryo implantation after transfer. The study cohort comprised 48 subjects, as detailed in Table 2. Out of 48 subjects, 30 (62.5%) had implantation after FET. Subsequently there were 24 clinical pregnancies (50%), 4 miscarriages (8.3%) and 3 biochemical pregnancies (6.3%). At that time of data analysis, there have been 13 live births (27.1%) and 11 ongoing pregnancies (22.9%). For the purpose of analysis, patients with an ongoing pregnancy and live birth were grouped together and are denoted as the ongoing pregnancy group. Patients with an ongoing pregnancy (n=24) were compared to those that did not achieve implantation after FET (n=17). There were no significant differences in age (35.17±4.57 vs. 33.71±4.38 years, p=0.31) or BMI (27.91±6.85 vs. 27.24±7.45 kg/m², p=0.77) between the two groups. The duration of estrogen treatment and cycle day at the start of progesterone were also similar between groups (15.00±3.53 vs. 15.00±4.75 days, p=0.97; 13.63±2.68 vs. 12.71±1.53, p=0.21, respectively). Endometrial thickness at the start of progesterone did not significantly differ (10.63±1.69 mm vs. 10.24±1.44 mm, p=0.44). Preimplantation genetic testing for aneuploidy (PGT-A) was performed in 62.5% of ongoing pregnancies compared to 76.5% in the non-pregnant group (p=0.50). Notably, all patients underwent single embryo transfer.

To retrieve a uterine cavity fluid sample, cells are obtained from the micro-fluidic compartment, on top of the epithelial cells in the uterine cavity. For example, under physiological conditions, microliter amounts of fluid are secreted by endometrial glands. A catheter is placed in the uterine cavity and cells are collected within the micro-fluid (microliter volumes) on the surface of the epithelial cells in the uterine cavity.

The etiology of infertility showed a trend toward significance (p=0.08). Among patients with ongoing pregnancies, the most common causes for infertility were tubal factor (29.2%), unexplained infertility (25.0%), and male factor (20.8%). In contrast, unexplained infertility was the most prevalent cause in the non-pregnant group (41.2%), followed by tubal factor (17.6%) and male factor (17.6%). Endometriosis and polycystic ovarian syndrome (PCOS) were comparable between groups (4.2% vs. 5.9% for endometriosis, and 8.3% vs. 11.8% for PCOS, respectively). One patient (5.9%) in the non-pregnant group had infertility classified as other due to a carrier of a monogenic disease undergoing PGT-M, while none were reported in the ongoing pregnancy group.

Uterine cavity fluid was collected from the whole uterus of 48 females who underwent FET. Uterine cavity fluid samples were collected on the day of embryo transfer, and pelleted cells stained with live/dead kit and anti-CD45 and analyzed on FlowJo software. (n=40+/−SD unpaired t-test (two-tailed), *p<0.05).

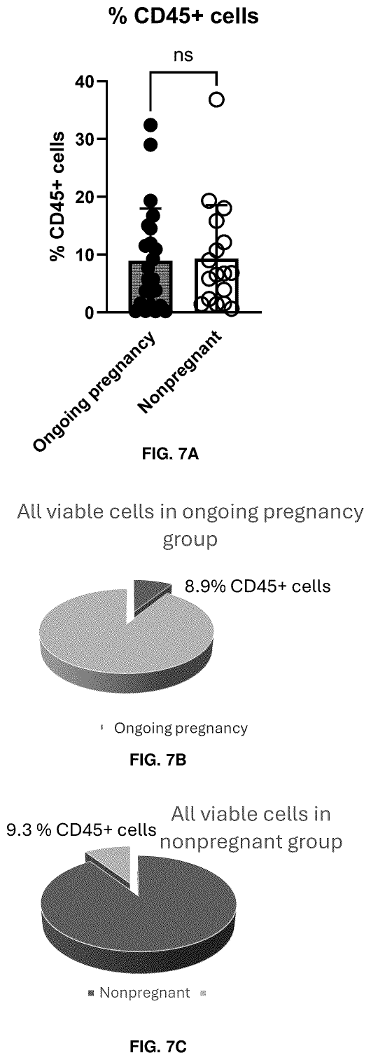

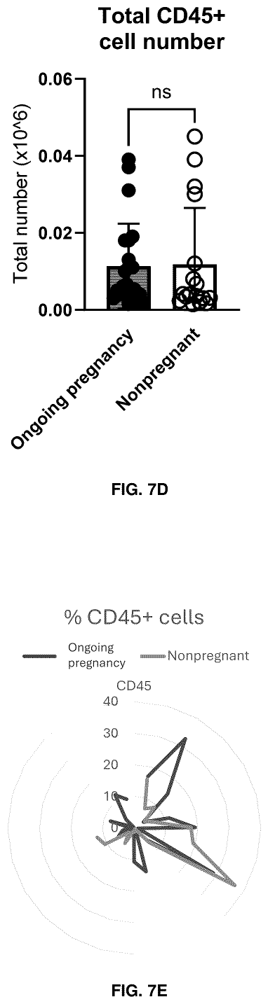

To determine frequency of hematopoietic immune cells within the collected uterine cavity fluid cells, cells were stained with anti-CD45 antibody. Live, CD45+ cells are present in the uterine cavity fluid at the time of FET and constitute around about 9% of all viable uterine cells collected, as seen in the pie charts of FIGS. 7B and FIG. 7C. There did not appear to be a statistically significant difference in the percentage and number of CD45+ cells between patients with an ongoing pregnancy and those without implantation (8.9%+/−9.0 SD vs. 9.3%+/−9.2 SD p=0.621; 0.0113×10⁶+/−0.0110 SD vs 0.0118×10⁶+/−0.0146 SD p=0.359), as seen in FIG. 7A or FIG. 7D. FIG. 7E depicts a radar chart of the summary of distribution of the percent of live, CD45+ cells in ongoing pregnancy and the non-pregnant groups as seen in FIGS. 7A-7D, where the orange line represents distribution in non-pregnant group and blue line represents the ongoing pregnancy group.

Comparison of uterine cavity fluid immune cell distribution between ongoing pregnancy and non-pregnant subjects

To explore the relationship between various immune cells at the time of FET and reproductive outcomes, subjects entering the study were divided according to their pregnancy outcome (ongoing pregnancy and non-pregnant). Uterine cavity fluid samples were collected on the day of embryo transfer, and pelleted cells stained with live/dead kit and anti-CD45, anti-CD3, anti-CD4, anti-CD8, anti-TCR GD, anti-CD25, anti-CD127, anti-CD19, anti-CD56, anti-CD14, anti-CD66b, and analyzed on FlowJo software. (n=40+/−SD unpaired t-test (two-tailed), *p<0.05).

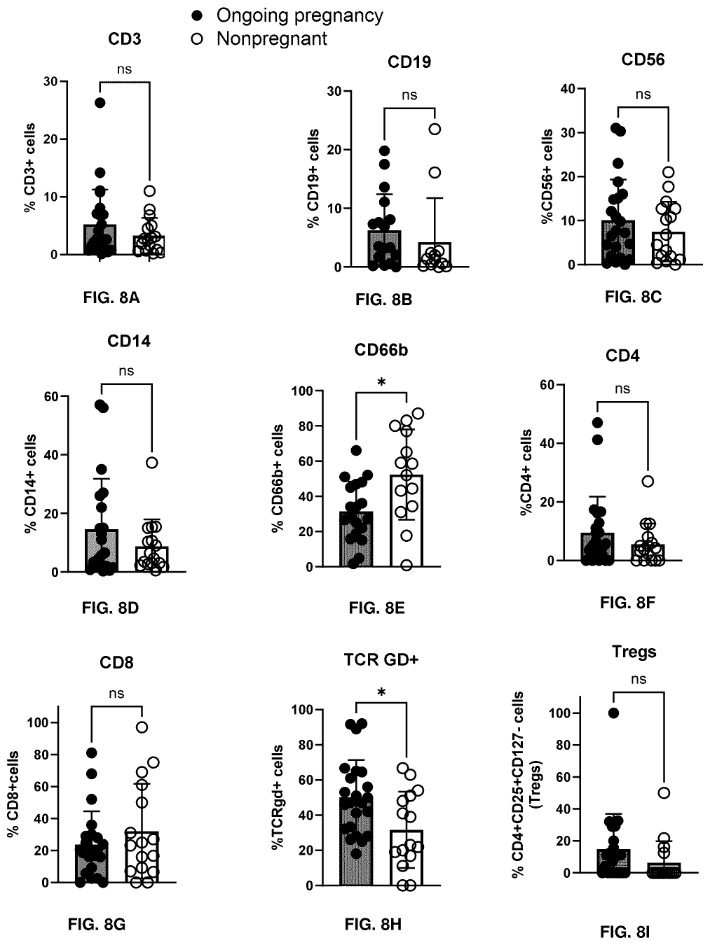

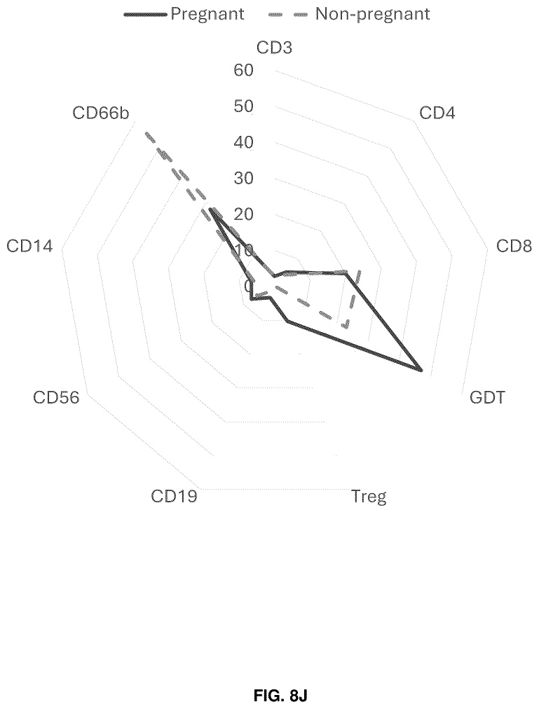

There were no significant differences in baseline data between the two groups, seen in FIG. 8A-8D, 8F-8G, and 81. There was no statistically significant difference in percentage of CD3 (5.23%+/−6.02 vs 3.27%+/−3.06; p=0.136), CD19 (6.27%+/−6.13 SD vs 4.22%+/−7.49 SD; p=0.136), CD56 (10.08%+/−9.28 SD vs 7.50%+/−6.77 SD; p=0.468), CD14 (14.60%+/−17.15 SD vs 8.79%+/−9.16 SD; p=0.688), CD4 (9.51%+/−12.27 SD vs 5.59%+/−7.01 SD; p=0.327), CD8 (23.68%+/−20.78 SD vs 32.10%+/−29.54; p=0.563), and CD4+CD25+CD127− (14.76%+/−22.16 SD vs 6.27%+/−13.56; p=0.059) between ongoing pregnancy and non-pregnant groups.

Analyzing the expressions of different immune-cell markers using flow cytometry, there was a significant increase in the percentage of gamma delta TCR+ cells (GDT cells) in the group of ongoing pregnancy compared to the non-pregnant group (50.15%+/−21.19 SD vs 31.64%+/−21.72 SD; p=0.0197), as seen in FIG. 8H. In contrast, the percentage of CD66b+ cells (neutrophils) increased in the non-pregnant group compared to the group of ongoing pregnancy (52.36%+/−25.62 SD vs 31.44%+/−16.97SD; p=0.0143), as seen in FIG. 8E. The radar chart of FIG. 8J shows a summary of the distribution of different immune cells in ongoing pregnancy and non-pregnant groups seen in FIGS. 8A-81, where the dotted line represents distribution of the non-pregnant group and solid line represents the ongoing pregnancy group.

### Uterine Gamma Delta TCR+ and CD66b+ Cells As Potential Biomarkers for Predicting Pregnancy Outcomes After Fet Through the Analysis of ROC (Receiver Operating Characteristic) Curves

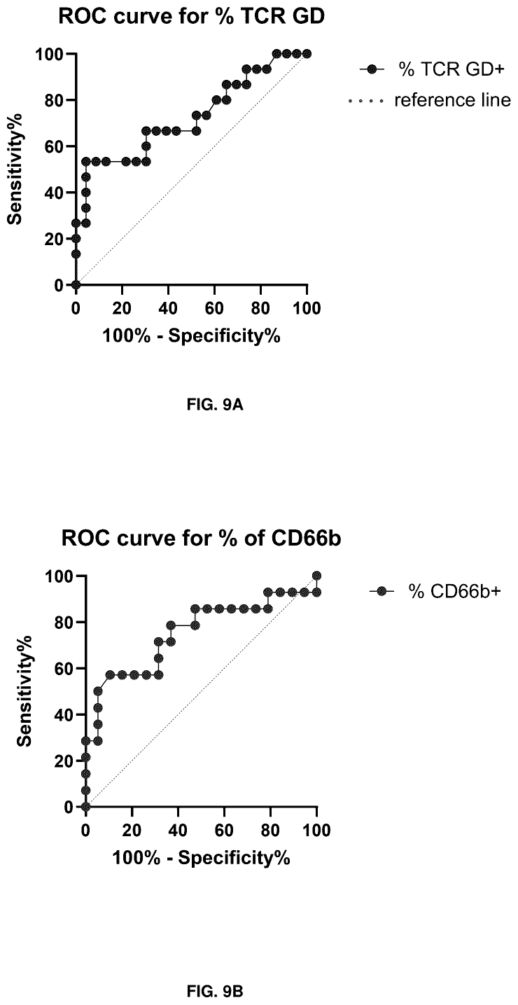

The percentages of gamma delta TCR+ and CD66b+ cells in the uterine lining of patients undergoing FET were analyzed. ROC analysis, seen in FIG. 9A, showed that the optimal cutoff value for gamma delta TCR+ cells is >24% and classify subjects below this value as a higher risk for not getting pregnant using the derivation set. Specificity for this value is very high, 95%, meaning that only 5% of pregnant patients will have less than 24% of uterine cavity fluid gamma delta TCR+ cells. To quantify the overall ability of the uterine cavity fluid cell-frequency test to discriminate between those individuals that will achieve an ongoing pregnancy and those without pregnancy, area under a ROC curve (AUC) statistic was used. An AUC of 0.72 with 95% confidence interval (CI) 0.5504 to 0.8989 for gamma delta TCR+ cells was found, which represents the probability of 72% that a randomly selected non-pregnant patient undergoing FET will have a lower test result than a randomly selected pregnant patient. Importantly, higher AUC indicates better predictive ability with p value=0.0206 which overall confirms that our test discriminates between no implantation and ongoing pregnancy.

It was discovered that the optimal cutoff value for CD66b+ cells is >39.60% and classify patients above this value as a higher risk for implantation failure with specificity of 68.42% (95% CI 46.01% to 84.64%). In addition, it was discovered that the AUC is 0.75 (95% CI 0.5681 to 0.9319) with p=0.0154, confirming overall ability of CD66b test to discriminate between non-pregnant and ongoing pregnancy, with a probability of 75% that a randomly selected non-pregnant patient undergoing FET will have a higher CD66b test result than a randomly selected patient with an ongoing pregnancy at the time of the FET, as seen in FIG. 9B.

In some embodiments, the evaluation of percentage of uterine TCR GD+ and CD66b+ cells by ROC curve analysis may be used as a novel diagnostic pregnancy-predictor test for FET.

Overall, this is the first study to present the potential of immune cell profiles at the time of FET as predictive tools for IVF outcomes, highlighting the importance of uterine immune responses in pregnancy success.

Confirming the overall ability of a CD66b test to discriminate between non- pregnant and ongoing pregnancy with a high probability, a colorimetric evaluation of CD66b expression in the lysed uterine cell samples was performed.

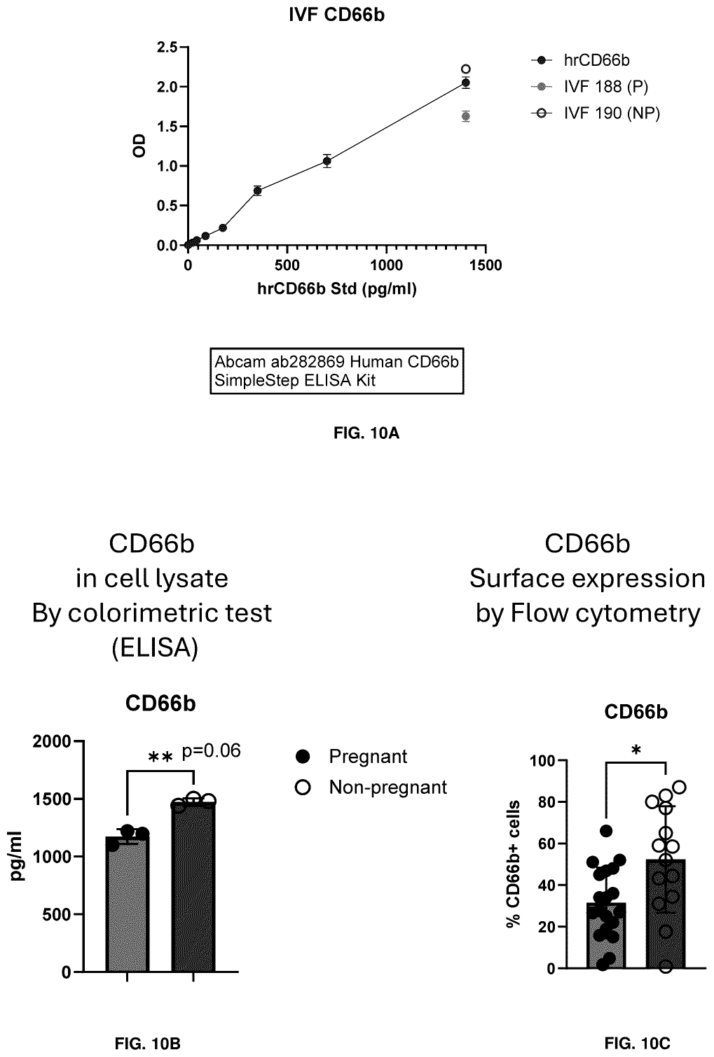

Human CD66b ELISA Kit (Abcam, ab282869) (a single-wash 90-min SimpleStep ELISA®) was used to quantify human CD66b in lysed uterine cell samples from pregnant and non-pregnant women with test sensitivity of 3.93 pg/ml. The assay uses a simple mix-wash-read protocol with just one incubation and one wash step and colorimetric sandwich ELISA-450 nm readout works on any standard plate reader. FIG. 10A depicts a standard curve of CD66b, where the background-subtracted data values (mean+/−SD) are graphed. FIG. 10B depicts a graph of the interpolated concentrations of CD66b in pregnant and non-pregnant lysed uterine cell samples. The concentrations of CD66b were measured in duplicates, interpolated from the CD66b standard curves and corrected for sample dilution. Undiluted samples are as follows: lysed pregnant uterine cells, and lysed non-pregnant uterine cells. The interpolated dilution factor corrected values are plotted (mean+/−SD, n=3). FIG. 10C depicts a graph of the percentage of uterine leukocyte cells (CD45+) that express CD66b on their surface within the lysed uterine cell samples. These results confirm that the increased level of CD66b can be measured in the non-pregnant uterine cell samples when compared to pregnant uterine cell samples (1474 pg/ml for non-pregnant vs 1173 pg/ml for pregnant) and are predictive of whether embryo implantation will be successful after frozen embryo transfer.

It is understood, therefore, that this invention is not limited to the particular embodiments disclosed but is intended to cover all modifications which are within the spirit and scope of the invention as defined by the appended claims; the above description, and/or shown in the attached drawings. Consequently, only such limitations as appear in the appended claims should be placed on the disclosure

### REFERENCES

1. Yu, M., et al., N-glycosylation of uterine endometrium determines its receptivity. J Cell Physiol, 2020. 235(2): p. 1076-1089.
2. Mahajan, N., Endometrial receptivity array: Clinical application. J Hum Reprod Sci, 2015. 8(3): p. 121-9.
3. Das, S. K., et al., Heparin-binding EGF-like growth factor gene is induced in the mouse uterus temporally by the blastocyst solely at the site of its apposition: a possible ligand for interaction with blastocyst EGF-receptor in implantation. Development, 1994. 120(5): p. 1071-83.
4. Fox, C., et al., Local and systemic factors and implantation: what is the evidence? Fertil Steril, 2016. 105(4): p. 873-84.
5. Khadem, N., et al., Association of IL-1 and TNF-alpha Levels in Endometrial Secretion and Success of Embryo Transfer in IVF/ICSI Cycles. Int J Fertil Steril, 2019. 13(3): p. 236-239.
6. Neves, A. R., et al., What is the clinical impact of the endometrial receptivity array in PGT-A and oocyte donation cycles? J Assist Reprod Genet, 2019. 36 (9): p. 1901-1908.
7. Berlanga, O., et al., How endometrial secretomics can help in predicting implantation. Placenta, 2011. 32 Suppl 3: p. S271-5.
8. Gemzell-Danielsson, K. and M. Hamberg, The effect of antiprogestin (RU 486) and prostaglandin biosynthesis inhibitor (naproxen) on uterine fluid prostaglandin F2 alpha concentrations. Hum Reprod, 1994. 9(9): p. 1626-30.
9. Li, T., et al., Cargo small non-coding RNAs of extracellular vesicles isolated from uterine fluid associate with endometrial receptivity and implantation success. Fertil Steril, 2021. 115(5): p. 1327-1336.
10. Ng, Y. H., et al., Endometrial exosomes/microvesicles in the uterine microenvironment: a new paradigm for embryo-endometrial cross talk at implantation. PLOS One, 2013. 8(3): p. e58502.
11. von Grothusen, C., et al., Uterine fluid microRNAs are dysregulated in women with recurrent implantation failure. Hum Reprod, 2022. 37(4): p. 734-746.
12. Kasvandik, S., et al., Uterine Fluid Proteins for Minimally Invasive Assessment of Endometrial Receptivity. J Clin Endocrinol Metab, 2020. 105(1).
13. Chen, X., et al., Measurement of uterine natural killer cell percentage in the periimplantation endometrium from fertile women and women with recurrent reproductive failure: establishment of a reference range. Am J Obstet Gynecol, 2017. 217(6): p. 680 e1-680 e6.
14. Li, F., et al., Potential Biomarkers and Endometrial Immune Microenvironment in Recurrent Implantation Failure. Biomolecules, 2023. 13(3).
15. Li, Y., et al., Evaluation of peripheral and uterine immune status of chronic endometritis in patients with recurrent reproductive failure. Fertil Steril, 2020. 113(1): p. 187-196 e1.
16. Quenby, S., et al., Pre-implantation endometrial leukocytes in women with recurrent miscarriage. Hum Reprod, 1999. 14(9): p. 2386-91.
17. Flemming, S., et al., Analysis of leukocyte transepithelial migration using an in vivo murine colonic loop model. JCI Insight, 2018. 3(20).
18. Liu, Y., et al., Human junction adhesion molecule regulates tight junction resealing in epithelia. J Cell Sci, 2000. 113 (Pt 13): p. 2363-74.
19. Mitchell, L. A., et al., Junctional adhesion molecule A promotes epithelial tight junction assembly to augment lung barrier function. Am J Pathol, 2015. 185(2): p. 372-86.
20. Baumgart, D. C. and W. J. Sandborn, Crohn's disease. Lancet, 2012. 380(9853): p. 1590-605
21. Ordas, I., et al., Ulcerative colitis. Lancet, 2012. 380(9853): p. 1606-19.
22. Harper, M. J., The implantation window. Baillieres Clin Obstet Gynaecol, 1992. 6(2): p. 351-71.
23. Zenclussen, A. C. and G. J. Hammerling, Cellular Regulation of the Uterine Microenvironment That Enables Embryo Implantation. Front Immunol, 2015. 6: p. 321.
24. Sauerbrun-Cutler, M. T., et al., Do endometrial natural killer and regulatory T cells differ in infertile and clinical pregnancy patients? An analysis in patients undergoing frozen embryo transfer cycles. Am J Reprod Immunol, 2021. 85(6): p. e13393.
25. Fu, B., et al., Natural killer cells promote immune tolerance by regulating inflammatory TH17 cells at the human maternal-fetal interface. Proc Natl Acad Sci U S A, 2013. 110(3): p. E231-40.
26. Cheng, Y., et al., Transcriptomic analysis of mid-secretory endometrium reveals essential immune factors associated with pregnancy after single euploid blastocyst transfer. Am J Reprod Immunol, 2023. 89(3): p. e13672.
27. Fan, X., et al., CD19 and intraglandular CD163-positivity as prognostic indicators of pregnancy outcome in CD138-negative women with a previous fresh-embryo-transfer failure. J Reprod Immunol, 2021. 147: p. 103362.
28. Sudoma, I., et al., Immune phenotype of the endometrium in patients with recurrent implantation failures after the transfer of genetically tested embryos in assisted reproductive technology programs. J Reprod Immunol, 2023. 157: p. 103943.
29. Von Woon, E., et al., Number and function of uterine natural killer cells in recurrent miscarriage and implantation failure: a systematic review and meta-analysis. Hum Reprod Update, 2022. 28(4): p. 548-582.
30. Robertson, S. A., A. S. Care, and L. M. Moldenhauer, Regulatory T cells in embryo implantation and the immune response to pregnancy. J Clin Invest, 2018. 128(10): p. 4224-4235.
31. Tu, W., et al., Association between Peripheral CD19+ B Cells and Reproductive Outcome in Women with Recurrent Implantation Failure. Clin Lab, 2020. 66(1).
32. Guzman-Genuino, R. M., et al., Uterine B Cells Exhibit Regulatory Properties During the Peri-Implantation Stage of Murine Pregnancy. Front Immunol, 2019. 10: p. 2899.
33. Lima, J., et al., Characterization of B cells in healthy pregnant women from late pregnancy to post-partum: a prospective observational study. BMC Pregnancy Childbirth, 2016. 16(1): p. 139.
34. Huang, C., Y. Zeng, and W. Tu, The role of gammadelta-T cells during human pregnancy. Am J Reprod Immunol, 2017. 78(2).
35. Szekeres-Bartho, J., et al., The role ofγ/δT cells in the feto-maternal relationship. Seminars in Immunology, 2001. 13(4): p. 229-233.
36. Poggi, A. and M. R. Zocchi, gammadelta T Lymphocytes as a First Line of Immune Defense: Old and New Ways of Antigen Recognition and Implications for Cancer Immunotherapy. Front Immunol, 2014. 5: p. 575.
37. Chang, R. Q., et al., Decidual RANKL/RANK interaction promotes the residence and polarization of TGF-beta1-producing regulatory gammadelta T cells. Cell Death Dis, 2019. 10(2): p. 113.
38. Aluvihare, V. R., M. Kallikourdis, and A. G. Betz, Regulatory T cells mediate maternal tolerance to the fetus. Nat Immunol, 2004. 5(3): p. 266-71.
39. Ali, S. B., et al., The role of immunological testing and intervention in reproductive medicine: A fertile collaboration? Am J Reprod Immunol, 2018. 79(3).
40. Tilburgs, T. and J. L. Strominger, CD8+ effector T cells at the fetal-maternal interface, balancing fetal tolerance and antiviral immunity. Am J Reprod Immunol, 2013. 69(4): p. 395-407.
41. Hardardottir, L., et al., The New Old CD8+ T Cells in the Immune Paradox of Pregnancy. Front Immunol, 2021. 12: p. 765730.
42. Tilburgs, T., et al., Human decidual tissue contains differentiated CD8+ effector-memory T cells with unique properties. J Immunol, 2010. 185(7): p. 4470-7.
43. Zeng, W., et al., Deep Surveying of the Transcriptional and Alternative Splicing Signatures for Decidual CD8(+) T Cells at the First Trimester of Human Healthy Pregnancy. Front Immunol, 2018. 9: p. 937.
44. White, H.D., et al., CD3+ CD8+ CTL activity within the human female reproductive tract: influence of stage of the menstrual cycle and menopause. J Immunol, 1997. 158(6): p. 3017-27.
45. Slutsky, R., et al., Exhausted and Senescent T Cells at the Maternal-Fetal Interface in Preterm and Term Labor. J Immunol Res, 2019. 2019: p. 3128010.
46. Witko-Sarsat, V., et al., Neutrophils: molecules, functions and pathophysiological aspects. Lab Invest, 2000. 80(5): p. 617-53.
47. Amsalem, H., et al., Identification of a novel neutrophil population: proangiogenic granulocytes in second-trimester human decidua. J Immunol, 2014. 193(6): p. 3070-9.
48. Hebeda, C. B., et al., Neutrophil depletion in the pre-implantation phase impairs pregnancy index, placenta and fetus development. Front Immunol, 2022. 13: p. 969336.
49. McMaster, M. T., S. K. Dey, and G. K. Andrews, Association of monocytes and neutrophils with early events of blastocyst implantation in mice. J Reprod Fertil, 1993. 99(2): p. 561-9.
50. Mohapatra, S. K., et al., Implantation associated changes in expression profile of indoleamine-2, 3-dioxygenase 1, Th1-Th2 cytokines and interferon-stimulated genes on neutrophils and peripheral blood mononuclear cells of crossbred cows. J Reprod Immunol, 2020. 142: p. 103188.
51. Mizugishi, K., et al., Sphingolipid pathway regulates innate immune responses at the fetomaternal interface during pregnancy. J Biol Chem, 2015. 290(4): p. 2053-68.
52. Petroff, M. G., et al., Decidual macrophages are potentially susceptible to inhibition by class la and class lb HLA molecules. J Reprod Immunol, 2002. 56(1-2): p. 3-17.
53. Heikkinen, J., et al., Phenotypic characterization of human decidual macrophages. Clin Exp Immunol, 2003. 131(3): p. 498-505.
54. Mclntire, R. H. and J. S. Hunt, Antigen presenting cells and HLA-G—a review. Placenta, 2005. 26 Suppl A: p. S104-9.
55. Fest, S., et al., Trophoblast-macrophage interactions: a regulatory network for the protection of pregnancy. Am J Reprod Immunol, 2007. 57(1): p. 55-66.
56. Helige, C., et al., Distribution of decidual natural killer cells and macrophages in the neighbourhood of the trophoblast invasion front: a quantitative evaluation. Hum Reprod, 2014. 29(1): p. 8-17.
57. Billiau, A., Interferon-gamma: biology and role in pathogenesis. Adv Immunol, 1996. 62: p. 61-130.
58. Tau, G. and P. Rothman, Biologic functions of the IFN-gamma receptors. Allergy, 1999. 54(12): p. 1233-51.
59. Chaudhari-Kank, M. S., et al., Comparison of CD9 & CD146 markers in endometrial stromal cells of fertile & infertile females. Indian J Med Res, 2018. 147(6): p. 552-559.
60. Salama, K. M., M. K. Alloush, and R. M. Al Hussini, Are the cytokines TNF alpha and IL 1Beta early predictors of embryo implantation? Cross sectional study. J Reprod Immunol, 2020. 137: p. 102618.
61. Kaislasuo, J., et al., IL-10 to TNFalpha ratios throughout early first trimester can discriminate healthy pregnancies from pregnancy losses. Am J Reprod Immunol, 2020. 83(1): p. e13195.

TABLE 1
Patient Demographics for Example 1.

| | Total (n = 24) |
|---|---|
| Age (years) | 34.75 +/− 4.09 |
| BMI (kg/m²) | 29.33 +/− 7.85 |
| Days on estrogen | 13.00 +/− 3.97 |
| Cycle day starting progesterone | 13.71 +/− 1.33 |
| Endometrial thickness at start of | 10.13 +/− 1.42 |
| progesterone (mm) | |
| ICSI n (%) | 23 (96) |
| Conventional IVF n (%) | 1 (4) |
| Number of embryos transferred | 1 (100) |

Data values are provided as n(%) or mean+/−standard deviation for continuous traits.

TABLE 2
Patient Demographics for Example 2.

| | Ongoing | Non- | |
|---|---|---|---|
| | pregnancy | Pregnancy | P |
| Characteristic | (n = 24) | (n = 17) | value |
| Age (years) | 35.17 +/− 4.57 | 33.17 +/− 4.38 | 0.31 |
| BMI (kg/m²) | 27.91 +/− 6.85 | 27.24 +/− 7.45 | 0.77 |
| Days on estrogen | 15.00 +/− 3.53 | 15.00 +/− 4.75 | 0.97 |
| Cycle day starting | 13.63 +/− 2.68 | 12.71 +/− 1.53 | 0.21 |
| progesterone | | | |
| Endometrial | 10.63 +/− 1.69 | 10.24 +/− 1.44 | 0.44 |
| thickness at | | | |
| start of | | | |
| progesterone (mm) | | | |
| PGT-An (%) | 15 (62.5%) | 13 (76.5%) | 0.50 |
| Single embryo | 24 (100%) | 17 (100%) | 1.0 |
| transfer n (%) | | | |
| Etiology of infertility | | | 0.08 |
| n (%) | | | |
| Endometriosis | 1 (4.2%) | 1 (5.9) | |
| Low ovarian reserve | 3 (12/5%) | 0 | |
| Unexplained | 6 (25.0%) | 7 (41.2%) | |
| Male factor | 5 (20.8%) | 3 (17.6%) | |
| Polycystic ovarian | 2 (8.3%) | 2 (11.8%) | |
| syndrome | | | |
| Tubal | 7 (29.2%) | 3 (17.6%) | |
| Other | 0 | 1 (5.9%) | |

Data values are provided as n(5) or mean+/−standard deviation for continuous traits.

1. A method for frozen embryo transfer(FET) in a subject, comprising:
acquiring, on the day of FET, a uterine cavity fluid sample from the subject;
performing one or more assays on the uterine cavity fluid sample to measure immune cell composition of the sample;
determining if the sample comprises a percent composition of immune cells above or below a threshold value of immune cell composition to provide a determined threshold value that indicates higher success of transfer implantation; and
transferring the frozen embryo into the subject if the sample comprises a percent composition of immune cells above a determined threshold value or a percent composition of immune cells below a threshold value that indicates higher success of transfer implantation.

2. The method of claim 1, wherein the uterine cavity fluid sample contains cells.

3. The method of claim 1, wherein acquiring a uterine cavity fluid sample from the subject comprises passing a catheter inside the uterine cavity.

4. A method for frozen embryo transfer(FET) in a subject, comprising:
acquiring, on the day of FET, a uterine cavity fluid sample from the subject;
performing one or more assays on the uterine cavity fluid sample to measure immune cell composition of the sample;
determining if the sample comprises a percent composition of gamma delta T cells above a threshold value and/or a percent composition of CD66b+ cells below a threshold value to determine a threshold value that indicates higher success of transfer implantation; and
transferring the frozen embryo into the subject if the sample comprises a percent composition of gamma delta T cells above a threshold value and/or a percent composition of CD66b+ cells below a threshold value that indicates higher success of transfer implantation.

5. The method of claim 4, wherein the uterine cavity fluid sample contains cells.

6. The method of claim 4, wherein acquiring a uterine cavity fluid sample from the subject comprises passing a catheter inside the uterine cavity.

7. The method of claim 4, wherein performing the assay comprises performing a colorimetric assay.

8. The method of claim 7, wherein the colorimetric assay comprises a colorimetric assay configured to determine expression levels of CD66b in the sample.

9. The method of claim 8, wherein if the level of CD66b detected in a colorimetric ELISA assay is 1200 pg/ml or below the transfer proceeds.

10. The method of claim 1, wherein the percent composition threshold value of gamma delta T Cells is about 24% or higher.

11. The method of claim 1, wherein the percent composition threshold value of CD66b is about 39% or less.

12. The method of claim 10, wherein if the sample comprises a percent composition of gamma delta TCR cells below 24% the frozen embryo is not transferred into the subject.

13. The method of claim 12, wherein the subject is administered an anti-inflammatory therapeutic to reduce the level of immune cells in the uterine cavity fluid.

14. The method of claim 13, wherein the anti-inflammatory therapeutic comprises prednisone, progesterone or other anti-inflammatory agents.

15. A system for frozen embryo transfer(FET) in a subject on day of embryo transfer, comprising:
a sample collection apparatus for collecting a uterine cavity fluid sample; and
one or more assays to determine percent composition of immune cells within the sample collected from the subject on the day of transfer.

16. The system of claim 15, wherein the one or more assays is configured to determine the percent composition of immune cells in the sample.

17. The system of claim 16, wherein the immune cells are gamma delta T cells and/or CD66b+ cells.

18. The system of claim 17, wherein the one or more assays comprise a colorimetric assay configured to determine expression levels of CD66b in the sample.

19. The system of claim 15, wherein the sample collection apparatus is a catheter.

20. The method of claim 10, wherein if the sample comprises a percent composition of CD66b cells above 39%, the frozen embryo is not transferred into the subject.
